(19) United States
(12) Patent Application Publication
(10) Pub. No.: US 2013/0256543 A1
(43) Pub. Date: Oct. 3, 2013

(54) DIGITAL X-RAY DETECTION HAVING AT LEAST ONE TRUNCATED CORNER

(71) Applicants: Paul Richard Granfors, Berkeley, CA (US); John Robert Lamberty, Oconomowoc, WI (US); German Guillermo Vera, Menomonee Falls, WI (US); Nicholas Ryan Konkle, Waukesha, WI (US); Habib Vafi, Brookfield, WI (US)

(72) Inventors: Paul Richard Granfors, Berkeley, CA (US); John Robert Lamberty, Oconomowoc, WI (US); German Guillermo Vera, Menomonee Falls, WI (US); Nicholas Ryan Konkle, Waukesha, WI (US); Habib Vafi, Brookfield, WI (US)

(73) Assignee: General Electric Company, Schenectady, NY (US)

(21) Appl. No.: 13/436,181

(22) Filed: Mar. 30, 2012

(51) Int. Cl. G01T 1/24; G01N 23/04

(52) U.S. Cl. 250/370.09; 378/62

(57) ABSTRACT

In one embodiment, a digital X-ray detector includes a plurality of pixel regions. Each pixel region includes one or more photodiodes. The plurality of pixel regions form a detector panel having at least one corner truncated with respect to a rectangle to form a rounded shape or greater than four-sided polygon.

### BACKGROUND OF THE INVENTION

The subject matter disclosed herein relates to digital imaging systems, and, more specifically, to digital X-ray detectors having at least one truncated corner with respect to a rectangle.

Digital X-ray imaging systems are becoming increasingly widespread for producing digital data which can be reconstructed into useful radiographic images. In current digital X-ray imaging systems, radiation from a source is directed toward a subject, typically a patient in a medical diagnostic application. A portion of the radiation passes through the patient and impacts a detector. The surface of the detector converts the radiation to light photons that are sensed. The detector is divided into a matrix of discrete picture elements or pixels, and encodes output signals based upon the quantity or intensity of the radiation impacting each pixel region. Because the radiation intensity is altered as the radiation passes through the patient, the images reconstructed based upon the output signals provide a projection of the patient's tissues similar to those available through conventional photographic film techniques.

Digital X-ray imaging systems are particularly useful due to their ability to collect digital data which can be reconstructed into the images required by radiologists and diagnosing physicians, and stored digitally or archived until needed. In conventional film-based radiography techniques, actual films were prepared, exposed, developed and stored for use by the radiologist. While the films provide an excellent diagnostic tool, particularly due to their ability to capture significant anatomical detail, they are inherently difficult to transmit between locations, such as from an imaging facility or department to various physician locations. The digital data produced by direct digital X-ray systems, on the other hand, can be processed and enhanced, stored, transmitted via networks, and used to reconstruct images which can be displayed on monitors and other soft copy displays at any desired location.

Similar advantages are offered by digitizing systems which convert conventional radiographic images from film to digital data.

Despite their utility in capturing, storing and transmitting image data, digital X-ray systems are still overcoming a number of challenges. For example, X-ray systems may be employed for a range of different types of examination, including radiographic and fluoroscopic imaging that may be useful for surgical applications. Some current digital X-ray systems employ X-ray detectors with arrays of photodiodes and thin film transistors beneath an X-ray scintillator. Incident X-rays interact with the scintillator to emit light photons which are absorbed by the photodiodes, creating electron-hole pairs. The diodes, which are initially charged with several volts of reverse bias, are thereby discharged in proportion to the intensity of the X-ray illumination. The thin film transistor switches associated with the diodes are then activated sequentially, and the diodes are recharged through charge sensitive circuitry, with the charge needed for this process being measured.

Many current X-ray digital detectors of this type utilize arrays of square pixels arranged in rows and columns. Accordingly, such detectors are often packaged and utilized in rectangular or square configurations. While such shapes may be useful for certain applications, a variety of applications, such as surgical applications, may utilize only a small area of the rectangular detector because the desired shape of the generated image must conform to an alternate shape, such as a circle or an oval. Accordingly, the configuration of many current X-ray detectors may result in one or more unutilized portions of the detector, thus reducing the efficiency of the X-ray system and contributing to the monetary cost of such systems.

### BRIEF DESCRIPTION OF THE INVENTION

In accordance with one embodiment, a digital X-ray detector includes a plurality of pixel regions. Each pixel region includes one or more photodiodes. The plurality of pixel regions form a detector panel having at least one corner truncated with respect to a rectangle to form a rounded shape or greater than four-sided polygon.

In accordance with another embodiment, a digital x-ray system includes a plurality of pixel regions arranged to define an image matrix having at least one corner truncated with respect to a rectangle to form a rounded shape or greater than four-sided polygon. The system also includes enable circuitry coupled to one or more photodiodes in each pixel region for enabling readout of the one or more photodiodes in each pixel region and readout circuitry coupled to the one or more photodiodes in each pixel region for reading out data from the one or more photodiodes.

In accordance with a third embodiment, a digital X-ray detector includes a detector panel having a plurality of pixel regions disposed on a first side of the detector panel. Each pixel region includes a one or more photodiodes. The detector also includes enable circuitry disposed on a second side of the detector panel opposite the first side and coupled to the first and second photodiodes of each pixel region for enabling readout of the first and second photodiodes. The system further includes readout circuitry disposed on the second side of the detector panel and coupled to the first and second photodiodes of each pixel region for reading out data from the first and second photodiodes. Additionally, the system includes a plurality of vias disposed in the detector panel and adapted to receive a plurality of conductors that communicatively couple the photodiodes of each pixel region to the enable and readout circuitry.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical overview of a digital X-ray imaging system, in accordance with aspects of the present technique;

FIG. 2 is a diagrammatical representation of certain embodiments of the functional circuitry for producing image data in a detector of the system of FIG. 1 to produce image data for reconstruction;

FIG. 3 is a schematic of an illustrative portion of an X-ray detector panel having a stepped perimeter in accordance with an embodiment;

FIG. 4 is a schematic of an illustrative portion of an X-ray detector panel having a polygonal perimeter in accordance with an embodiment;

FIG. 5 is a schematic illustrating a combined data and scan module in accordance with an embodiment;

FIG. 6 is a schematic of an illustrative portion of an X-ray detector panel having a plurality of conductors routed along a perimeter of the panel in accordance with an embodiment;

FIG. 7 is a schematic of an illustrative portion of an X-ray detector panel having a curved pixel array in accordance with an embodiment;

FIG. 8 is a schematic of an illustrative portion of a rounded X-ray detector panel in accordance with an embodiment; and

FIG. 9 is a schematic illustrating routing of conductors through vias disposed in an X-ray detector panel in accordance with an embodiment.

### DETAILED DESCRIPTION OF THE INVENTION

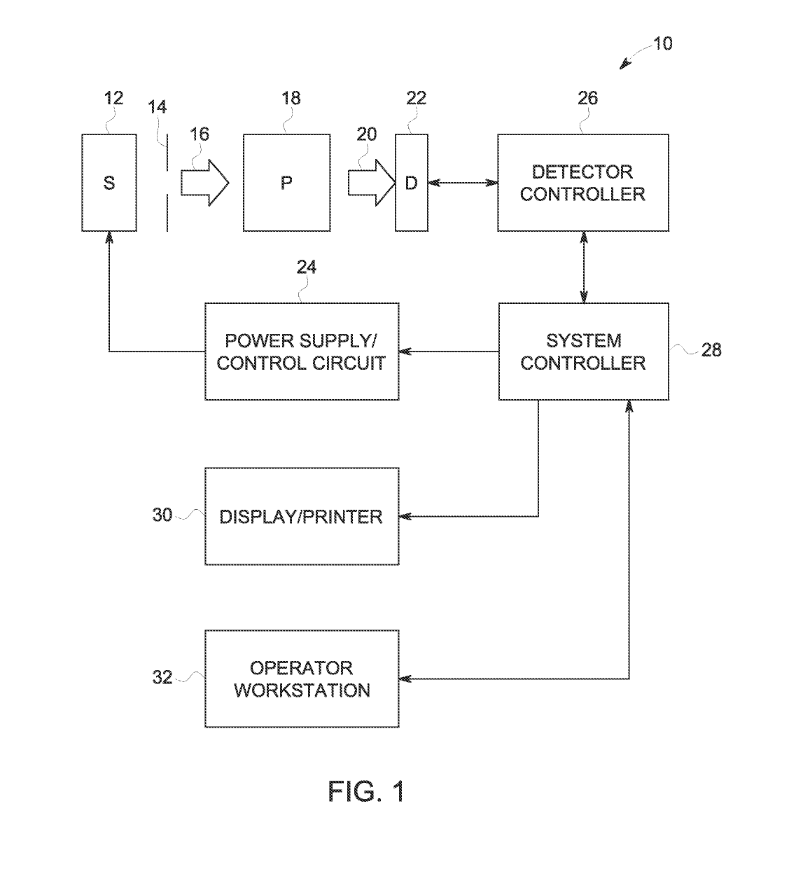

FIG. 1 illustrates diagrammatically an imaging system 10 for acquiring and processing discrete pixel image data. In the illustrated embodiment, a system 10 is a digital X-ray system designed both to acquire original image data, and to process the image data for display in accordance with the present technique. Throughout the following discussion, however, while basic and background information is provided on the digital X-ray system used in medical diagnostic applications, it should be born in mind that aspects of the present techniques may be applied to digital detectors, including X-ray detectors, used in different settings (e.g., projection X-ray, computed tomography imaging, tomosynthesis imaging, etc.) and for different purposes (e.g., parcel, baggage, vehicle and part inspection, etc.).

In the embodiment illustrated in FIG. 1, imaging system 10 includes a source of X-ray radiation 12 positioned adjacent to a collimator 14. Collimator 14 permits a stream of radiation 16 to pass into a region in which a subject 18, such as a human patient 18, is positioned. A portion of the radiation 20 passes through or around the subject 18 and impacts a digital X-ray detector, represented generally at reference numeral 22. As described more fully below, detector 22 converts the X-ray photons received on its surface to lower energy photons, and subsequently to electric signals which are acquired and processed to reconstruct an image of the features within the subject 18.

Source 12 is controlled by a power supply/control circuit 24 which furnishes both power and control signals for examination sequences. Moreover, detector 22 is coupled to a detector controller 26 which commands acquisition of the signals generated in the detector. Detector controller 26 may also execute various signal processing and filtration functions, such as for initial adjustment of dynamic ranges, interleaving of digital image data, and so forth. Both power supply/control circuit 24 and detector controller 26 are responsive to signals from a system controller 28. In general, system controller 28 commands operation of the imaging system to execute examination protocols and to process acquired image data. In the present context, system controller 28 also includes signal processing circuitry, typically based upon a general purpose or application-specific digital computer, associated memory circuitry for storing programs and routines executed by the computer, as well as configuration parameters and image data, interface circuits, and so forth. In the embodiment illustrated in FIG. 1, system controller 28 is linked to at least one output device, such as a display or printer as indicated at reference numeral 30. The output device may include standard or special purpose computer monitors and associated processing circuitry. One or more operator workstations 32 may be further linked in the system for outputting system parameters, requesting examinations, viewing images, and so forth. In general, displays, printers, workstations, and similar devices supplied within the system may be local to the data acquisition components, or may be remote from these components, such as elsewhere within an institution or hospital, or in an entirely different location, linked to the image acquisition system via one or more configurable networks, such as the Internet, virtual private networks, and so forth.

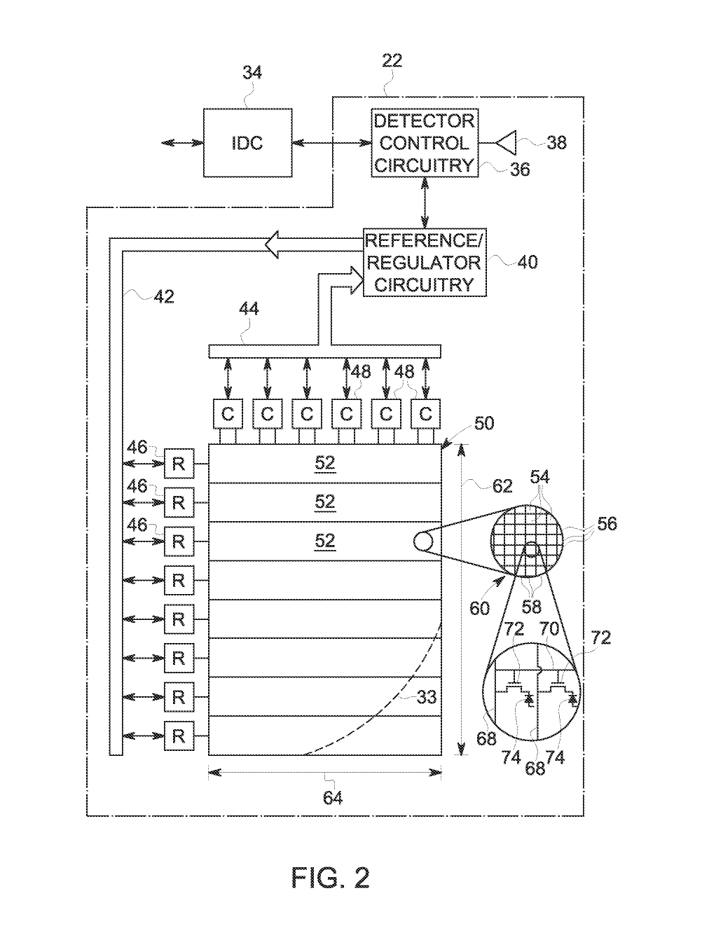

FIG. 2 is a diagrammatical representation of functional components of digital detector 22. FIG. 2 also represents an imaging detector controller or IDC 34 which will typically be configured within detector controller 26. IDC 34 includes a CPU or digital signal processor, as well as memory circuits for commanding acquisition of sensed signals from the detector. IDC 34 is coupled to detector control circuitry 36 within detector 22. The IDC 34 may be coupled to the detector control circuitry 36 via cables (e.g., fiber optic cables) or wirelessly. IDC 34 thereby exchanges command signals for image data within the detector during operation.

Detector control circuitry 36 receives DC power from a power source, represented generally at reference numeral 38. Detector control circuitry 36 is configured to originate timing and control commands for row drivers and column readout circuits used to transmit signals during data acquisition phases of operation of the system. Circuitry 36 therefore transmits power and control signals to reference/regulator circuitry 40, and receives digital image pixel data from circuitry 40.

In one embodiment, the detector 22 includes a scintillator that converts X-ray photons received on the detector surface during examinations to lower energy (light) photons. An array of photodetectors then converts the light photons to electrical signals which are representative of the number of photons or the intensity of radiation impacting individual pixel regions of the detector surface. As described below, readout electronics convert the resulting analog signals to digital values that can be processed, stored, and displayed, such as in a display 30 or a workstation 32 following reconstruction of the image. In a presently disclosed embodiment, the array of photodetectors is formed on a single base of amorphous silicon. The array elements or pixel regions are organized in rows and columns, with each pixel region consisting of one or more photodiodes. For example, in the illustrated embodiment, each pixel region includes first and second photodiodes. However, the illustrated embodiment is merely an example, and in other embodiments, any number of desired photodiodes may be utilized. Each photodiode has an associated thin film transistor. The cathode of each diode is connected to the source of the transistor, and the anodes of all diodes are connected to a negative bias voltage. The gates of the transistors in each row are connected together and the row electrodes are connected to the scanning electronics described below. The drains of the transistors in a column are connected together and an electrode of each column is connected to readout electronics.

It should be noted that a variety of arrangements of the array of pixel regions are presently contemplated in accordance with certain embodiments. In some embodiments, some or all of the pixels may be rectangular in shape, while in other embodiments, the pixels may be subject to a variety of implementation-specific configurations. Nevertheless, in some presently contemplated embodiments, the plurality of pixel regions form a detector panel having at least one corner truncated with respect to a rectangle to form a rounded shape or greater than four-sided polygon, as generally illustrated by line 33 in FIG. 2. Although only a single truncation is illustrated in FIG. 2, in other embodiments, additional truncations may be present in certain embodiments, as described in more detail below.

In the particular embodiment illustrated in FIG. 2, by way of example, a row bus 42 includes a plurality of conductors for enabling readout from various columns of the detector, as well as for disabling rows and applying a charge compensation voltage to selected rows, where desired. A column bus 44 includes additional conductors for reading out the columns while the rows are sequentially enabled. Row bus 42 is coupled to enable circuitry or a series of row drivers 46, each of which commands enabling of a series of rows in the detector. Similarly, readout circuitry 48 is coupled to column bus 44 for reading out all columns of the detector.

In the illustrated embodiment, row drivers 46 and readout circuitry 48 are coupled to a detector panel 50 which may be subdivided into a plurality of sections 52. Each section 52 is coupled to one of the row drivers 46, and includes a number of rows. Similarly, each column module 48 is coupled to a series of columns. The photodiode and thin film transistor arrangement mentioned above thereby define a series of pixel regions or discrete picture elements 54 which are arranged in rows 56 and columns 58. The rows and columns define an image matrix 60, having a height 62 and a width 64.

As also illustrated in FIG. 2, each photodiode of each pixel region 54 is generally defined at a row and column crossing, at which a row electrode or scan line 68 crosses a column electrode or data line 70. As mentioned above, a thin film transistor 72 is provided at each crossing location for each photodiode of each pixel region 54. As each row 56 is enabled by row drivers 46, signals from each photodiode 74 may be accessed via readout circuitry 48, and converted to digital signals for subsequent processing and image reconstruction. Here again, it should be noted that the particular arrangement of the pixel regions and the enable and readout circuitry may be subject to a variety of implementation-specific variations, as discussed in more detail below.

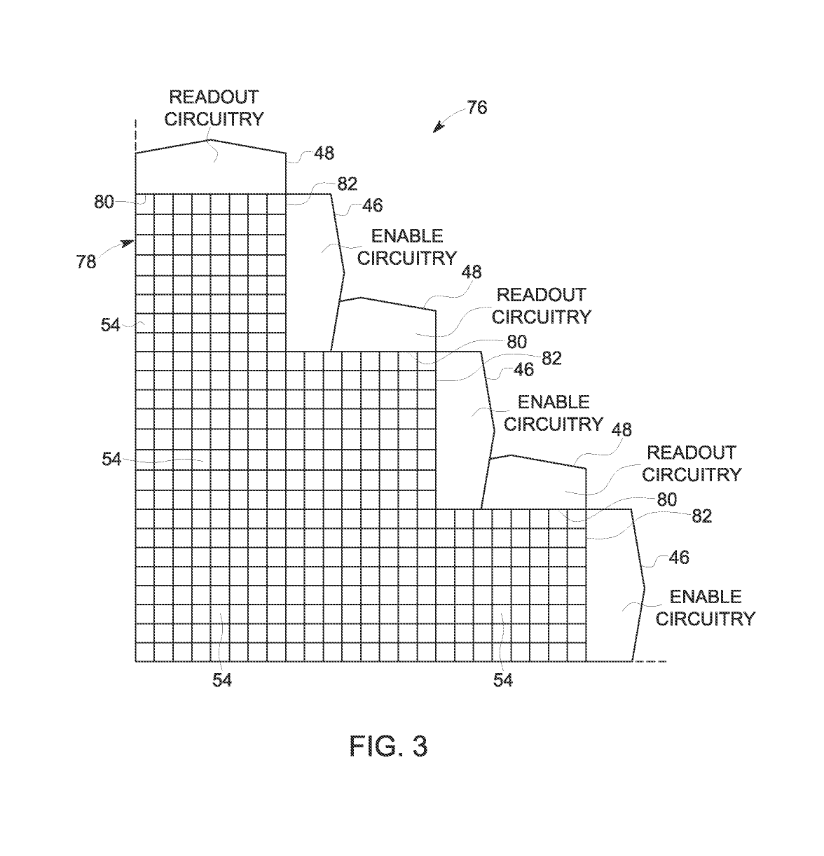

FIG. 3 is a schematic illustrating a portion 76 of an X-ray detector panel having stepped edges in accordance with one embodiment. As would be understood by one skilled in the art, the portion 76 of the X-ray panel that is shown represents a quarter of the presently contemplated X-ray detector panel. In the illustrated embodiment, an X-ray detector panel 78 includes a plurality of stepped edges, each having a first edge 80 and a second edge 82 that is approximately perpendicular to the first edge 80. As shown, readout circuitry 48 is coupled to each of the first edges 80, and enable circuitry 46 is coupled to each of the second edges 82 to enable readout of the photodiodes in each pixel region 54 and for reading out data from the photodiodes. The stepped edge feature of the foregoing embodiment may offer advantages over traditional systems that are rectangular in shape by reducing package size for applications in which the configuration of the desired image is circular.

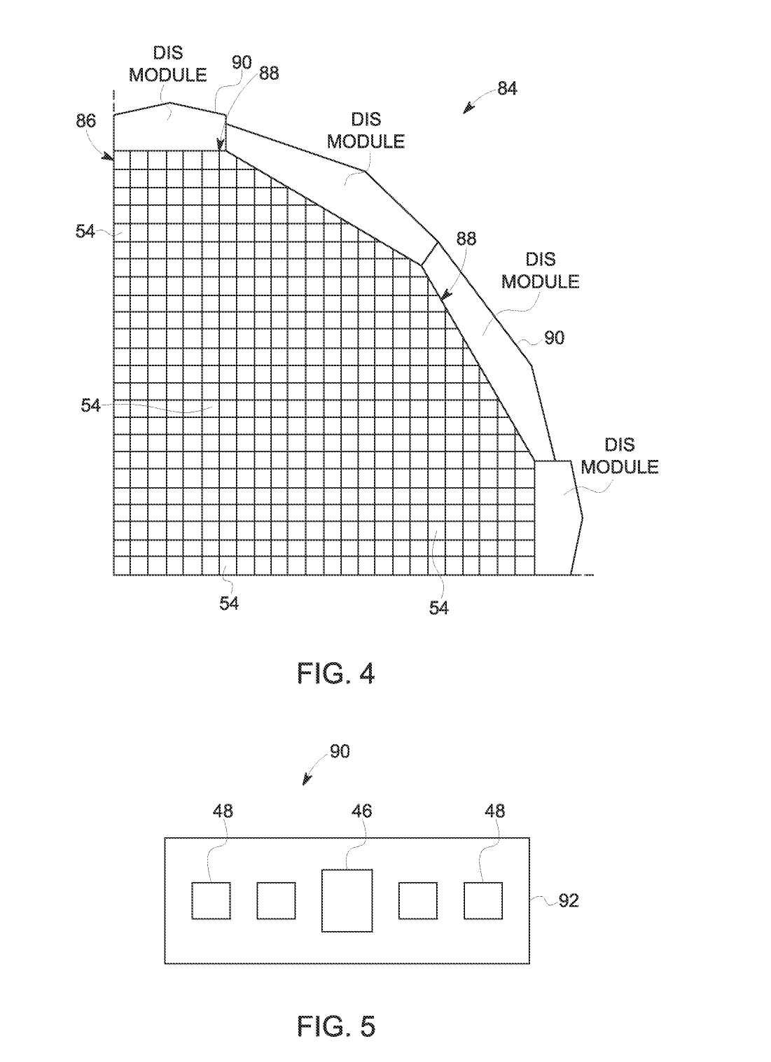

FIG. 4 is a schematic illustrating a portion 84 of a polygonal X-ray detector panel 86 having a plurality of angled edges 88 in accordance with one embodiment. Here again, as would be understood by one skilled in the art, the portion 84 of the X- ray panel 86 that is shown represents a quarter of the presently contemplated X-ray detector panel 86. In the illustrated embodiment, the X-ray detector panel 86 includes the plurality of angled edges 88 that form the perimeter of the panel 86. As such, in the illustrated embodiment, a series of corners of the panel are truncated with respect to a rectangle to form a polygon having more than four sides. In certain embodiments, the polygonal detector panel 86 may have five, six, seven, eight, or any other desired number of angled edges 88, as dictated by implementation-specific considerations. Additionally, it should be noted that the areas depicted as partial pixel areas may not be populated in certain embodiments, but may include conductors that couple to an associated data and scan module 90.

In the embodiment illustrated in FIG. 4, a plurality of combined data and scan modules 90 are disposed about the angled edges 88. Each data and scan module 90 includes both readout circuitry 48 as well as enable circuitry 46 disposed on a single base 92, as shown in FIG. 5. It should be noted that the scan and data modules 90 may include any desired number of co-packaged readout chips 48 and scan control chips 46. For example, in one embodiment, the combined module 90 may include approximately eight data readout chips 48 and approximately two scan control chips 46. However, in other embodiments, the quantity or ratios of the chips provided on each module 90 may depend on implementation-specific parameters, such as the quantity of angled edges 88 that form the perimeter of the detector panel 86. Nevertheless, in each embodiment, by providing both types of chips on the module base 92, each module 90 is capable of performing both readout and enabling functions for the columns and rows of pixels 54 with which the module 90 is associated.

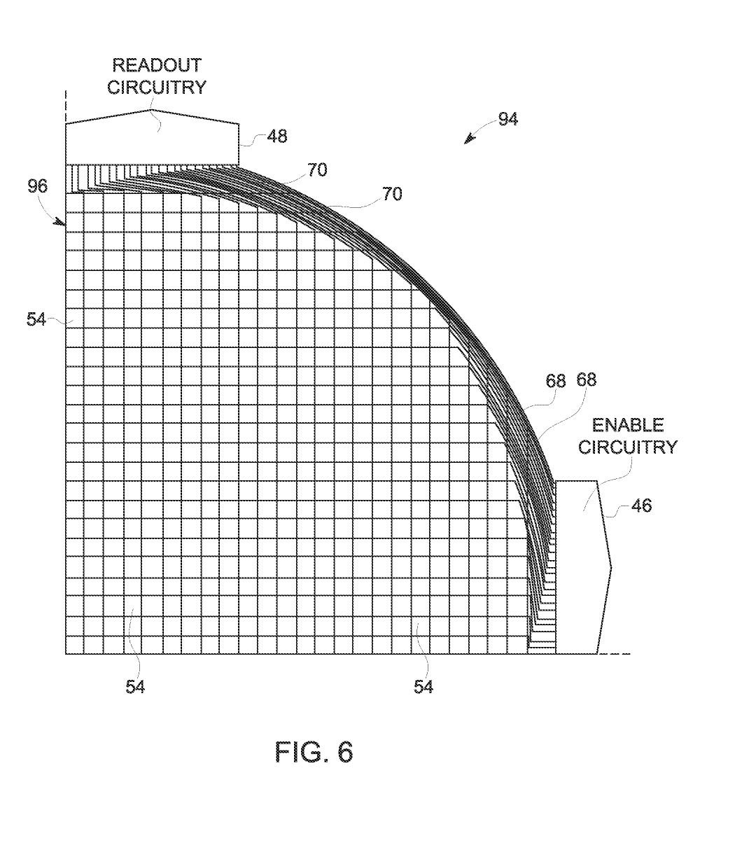

FIG. 6 is a schematic illustrating a portion 94 of a substantially rounded X-ray detector panel 96 having the scan and data conductors 68 and 70 routed about the perimeter of the panel 96 to the enable circuitry 46 and the readout circuitry 48, respectively. Here again, it should be noted that the partial pixel areas that are illustrated may not be populated but may still include the conductors 68 and 70. In this embodiment, the plurality of pixel regions 54 are arranged such that the configuration of the detector panel 96 is substantially rounded or circular. That is, the detector panel 96 includes four corners that are truncated with respect to a rectangle to form a substantially rounded shape. The foregoing feature may offer a variety of advantages over traditionally rectangular panels. For example, by providing a substantially circular image area, the demands of certain applications, such as surgical applications in which a circular image may be desirable, may be met while providing an increased packaging efficiency as compared to rectangular designs. Additionally, in this embodiment, the readout and enable circuitry modules 48 and 46 may only be needed on four sides of the detector panel 96, thus further improving packaging efficiency.

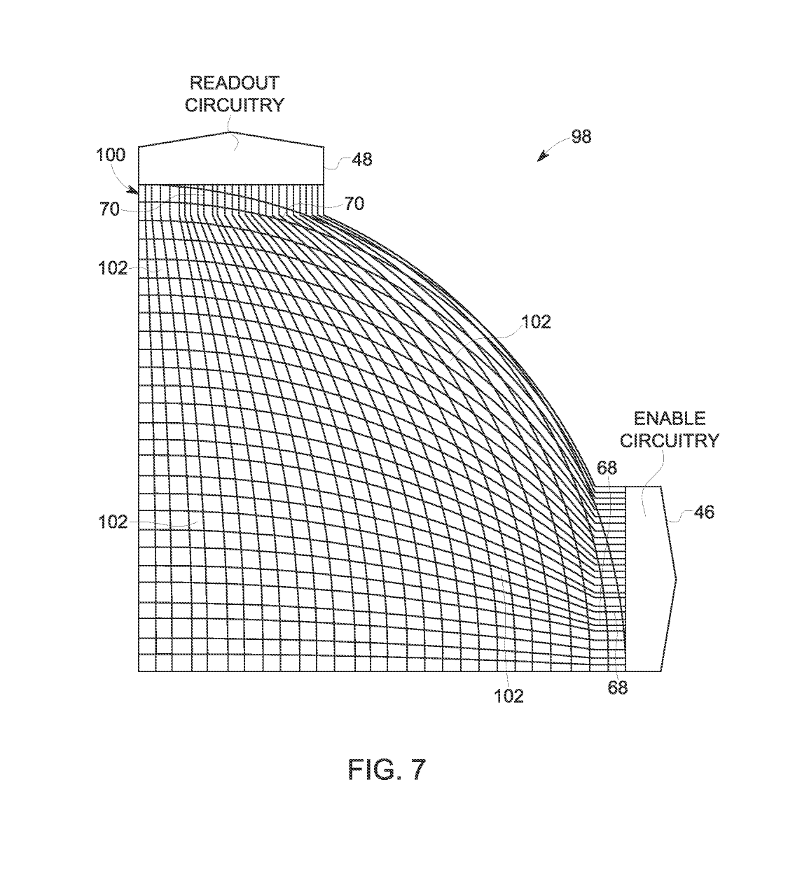

FIG. 7 is a schematic illustrating a portion 98 of a substantially curved X-ray detector panel 100 populated by a plurality of irregularly shaped pixels 102 connected to the readout circuitry 48 and the enable circuitry 46 via conductors 70 and 68, respectively. That is, in this embodiment, the pixels 102 may be subject to a variety of implementation-specific configurations suitable for forming the curved panel 100. As such, each pixel 102 may take on a different non-rectangular shape, as needed to form the desired curvature of the array. It should be noted that the particular size, shape, and location of each pixel 102 may be taken into account during image processing, for example, by detector controller 26 and/or system controller 28. here again, the packaging efficiency of the detector panel 100 may be improved as compared to rectangular designs that do not include at least one truncated corner.

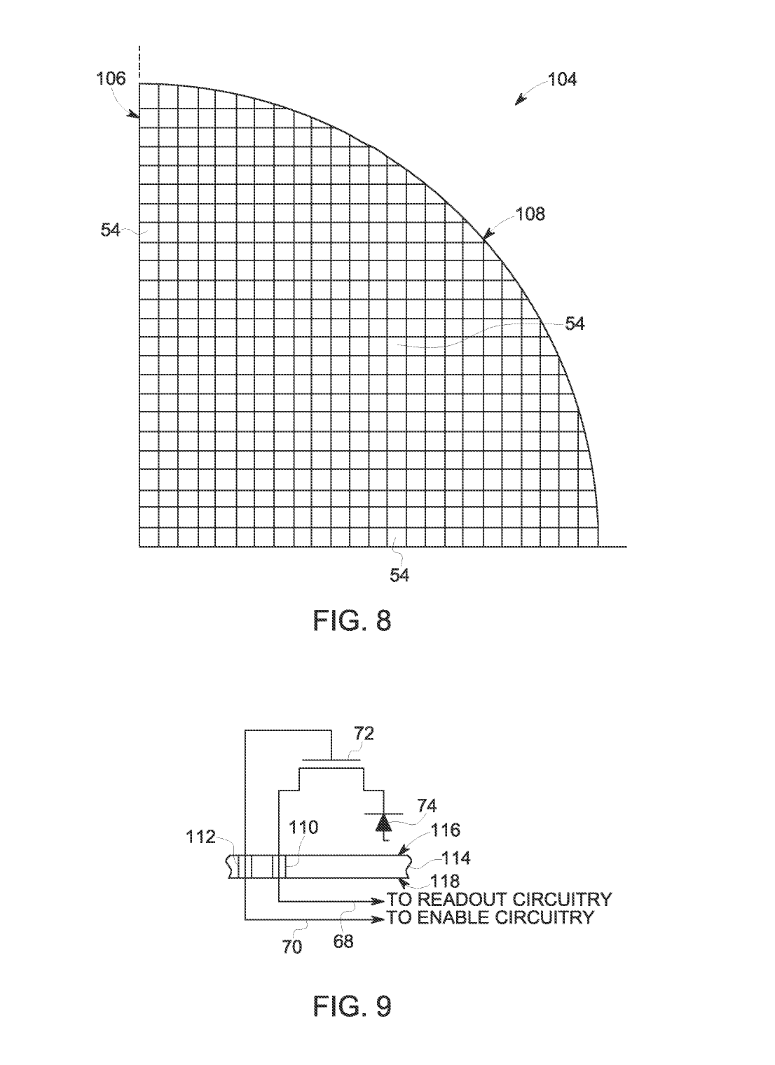

FIG. 8 is a schematic illustrating a portion 104 of a substantially circular X-ray detector panel 106 populated with pixels 54 and having a substantially continuous, circular edge 108. In this embodiment, the scan and data conductors 68 and 70 are routed through vias 110 and 112, respectively disposed in a wall 114 of the detector panel 106, as shown in FIG. 9. As illustrated, the vias 110 and 112 extend from a first side of the wall 114 to a second side of the wall 118, thus enabling the readout circuitry 48 and the enable circuitry 46 to be located on the back side 118 of the detector panel 106 with respect to the patient or object being imaged. The foregoing feature may enable scanning and reading to occur through the vias 110 and 112, thereby further improving packaging efficiency by positioning the electronics behind the detector panel 106.

It should be noted that in certain embodiments, additional layers may be provided on the second side 118 of the panel to facilitate routing of the conductors 68 and 70 to the appropriate circuitry. Additionally, it should be noted that the foregoing technique for routing the conductors 68 and 70 through vias disposed in the panel may be employed with panels of any desired shape, not limited to panels having truncated corners. For example, in other embodiments, vias may be provided in rectangular panels, stepped panels, polygonal panels, or any other configuration a detector panel may take on in a given application.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A digital X-ray detector comprising:
a plurality of pixel regions, each pixel region comprising one or more photodiodes, wherein the plurality of pixel regions form a detector panel comprising at least one corner truncated with respect to a rectangle to form a rounded shape or greater than four-sided polygon.

2. The detector of claim 1, comprising enable circuitry coupled to one or more photodiodes of each pixel region for enabling readout of the one or more photodiodes.

3. The detector of claim 1, comprising readout circuitry coupled to one or more photodiodes of each pixel region for reading out data from the one or more photodiodes.

4. The detector of claim 1, comprising a scan and data module coupled to the one or more photodiodes of each pixel region for enabling readout of the one or more photodiodes and for reading out data from the one or more photodiodes.

5. The detector of claim 1, wherein the detector panel comprises four corners truncated with respect to a rectangle to form a rounded or circular shape.

6. The detector of claim 5, comprising a plurality of vias disposed in the detector panel, each configured to receive a conductor configured to couple each pixel region to enable circuitry and readout circuitry.

7. The detector of claim 1, comprising a detector controller configured to control the plurality of pixel regions to control acquisition of signals generated in the detector.

8. The detector of claim 7, wherein the detector controller is further configured to process and filter the signals generated in the detector.

9. A digital x-ray system, comprising:
a plurality of pixel regions arranged to define an image matrix comprising at least one corner truncated with respect to a rectangle to form a rounded shape or greater than four-sided polygon;
enable circuitry coupled to one or more photodiodes in each pixel region for enabling readout of the one or more photodiodes in each pixel region; and
readout circuitry coupled to the one or more photodiodes in each pixel region for reading out data from the one or more photodiodes.

10. The system of claim 9, comprising a source of X-ray radiation configured to generate X-rays.

11. The system of claim 9, comprising detector control circuitry configured to originate timing and control commands for the enable circuitry and the readout circuitry to transmit signals during data acquisition.

12. The system of claim 11, wherein the detector control circuitry comprises an imaging detector controller comprising processing circuitry configured to process data from the photodiodes of each pixel region.

13. The system of claim 9, wherein a plurality of conductors couple the enable circuitry and the readout circuitry to the plurality of pixel regions, and wherein the plurality of conductors are routed from the plurality of pixel regions, around a perimeter of the plurality of pixel regions, and to the enable and readout circuitry.

14. The system of claim 9, comprising a plurality of conductors configured to couple the enable circuitry and the readout circuitry to the plurality of pixel regions, and a plurality of vias each configured to receive a conductor of the plurality of conductors to couple each pixel region to the enable and readout circuitry.

15. The system of claim 14, wherein the image matrix comprises four corners truncated with respect to a rectangle to form a rounded or circular shape.

16. A digital X-ray detector comprising:
a detector panel comprising a plurality of pixel regions disposed on a first side of the detector panel, each pixel region comprising one or more photodiodes;
enable circuitry disposed on a second side of the detector panel opposite the first side and coupled to the one or more photodiodes of each pixel region for enabling readout of the one or more photodiodes;
readout circuitry disposed on the second side of the detector panel and coupled to the one or more photodiodes of each pixel region for reading out data from the one or more photodiodes; and
a plurality of vias disposed in the detector panel and configured to receive a plurality of conductors that communicatively couple the photodiodes of each pixel region to the enable and readout circuitry.

17. The system of claim 16, wherein the detector panel comprises at least one corner truncated with respect to a rectangle to form a rounded or greater than four-sided polygon.

18. The system of claim 16, wherein the detector panel comprises four corners truncated with respect to a rectangle to form a rounded or circular shape.

19. The system of claim 16, wherein a scan and data module disposed on the second side of the detector panel comprises the enable circuitry and the readout circuitry.

20. The system of claim 16, comprising detector control circuitry configured to originate timing and control commands for the enable circuitry and the readout circuitry to transmit signals during data acquisition.
